(19) United States
(12) Patent Application Publication
(10) Pub. No.: US 2005/0197407 A1
(43) Pub. Date: Sep. 8, 2005

(54) Method and preparation for reducing irritation and/or inflammatory reaction in human skin

(75) Inventors: Joseph C. DiNardo, Raleigh, NC (US); Joseph A. Lewis II, Chesterfield, VA (US); Birgit Neudecker, Muenchen (DE); Falko Diedrich, Muenchen (DE); Eberhard Wieland, Stuttgart (DE)

Correspondence Address: DAVIDSON, DAVIDSON & KAPPEL, LLC, 485 SEVENTH AVENUE, 14TH FLOOR, NEW YORK, NY, 10018, US

(73) Assignee: PCR Technology Holdings, LC, Richmond, VA (US)

(21) Appl. No.: 11/057,364

(22) Filed: Feb. 14, 2005

Related U.S. Application Data
(60) Provisional application No. 60/544,359, filed on Feb. 13, 2004.

(51) Int. Cl. A61K 31/12

(52) U.S. Cl. 514/690

(57) ABSTRACT

A method for reducing an occurrence of irritation and/or inflammatory reaction in human skin includes applying a topical preparation to the skin. The preparation includes an amount of an agent effective to reduce an occurrence of irritation and/or inflammatory reaction in human skin. The agent includes idebenone or a derivative of idebenone.

Priority is claimed to provisional application 60/544,359, filed Feb. 13, 2004.

The present invention in general relates to human skin, and in particular relates to reduction of irritation and/or inflammatory reaction in human skin.

### BACKGROUND

A wide variety of compounds are formulated into cosmetics to, inter alia, improve the appearance of the skin, to beautify the skin, to treat skin conditions, or to render the skin less dry or more supple. Unfortunately, certain of these compounds may cause irritation and/or inflammatory reactions when they are topically applied onto the skin or mucous membranes.

It is known to this art to administer salicylic acid derivatives as keratolytic agents for treating acne; these are also useful anti-aging agents in cosmetic and/or dermatological compositions. Thus, FR-A-2,581,542 and EP-A-378,936 describe such salicylic acid compounds.

Salicylic acid derivatives are of great value, given their biological effects on the skin, in particular on the principal clinical signs of skin aging, namely, fine lines and wrinkles, disorganization of the “grain” of the skin, modification of the skin's complexion and loss of skin firmness and tonicity. However, administration of these derivatives presents a problem since they can cause stinging, itching and tautness after they have been applied, which may result in considerable discomfort. Users, and more particularly those having sensitive skin, are thus often discouraged from using these compounds. Sensitive skin and the signs which are characteristic of sensitive skin are described in EP-680,749.

The same also holds true for exfoliants such as alpha-hydroxy acids. Concerning these compounds, it has been proposed, in EP-413,528, to combine them with amphoteric species in order to decrease the discomfort that they may cause.

It is also widely described that some users of cosmetic products comprising surfactants, preservatives, fragrances, solvents or propellants complain that their skin is irritated after applying one or more of the above substrates thereto.

However, because of their irritant nature, these products are generally employed at very low doses. Formulation of these products in small amounts may, therefore, prove to be relatively disadvantageous with respect to administration of other active species which are less active, but which are formulated in greater amounts since they are less or not irritant.

It is also known to this art that the skin of certain individuals is irritated when it has been exposed to a specific or unusual chemical or physical environment, in particular when it is exposed to considerable variations in temperature, to wind or to a very polluted environment. Thus, the skin may react with itching, tautness or stinging.

In addition to chemicals which directly trigger skin irritation, some chemicals indirectly cause the skin to become more sensitive to other chemicals or environmental conditions which would not normally cause irritation. Many chemicals which act as skin “exfoliants” such as retinoids (e.g. tretinoin, retinol and retinal), carboxylic acids including alpha-hydroxy acids (e.g. lactic acid, glycolic acid), beta hydroxy acids (e.g. salicylic acid, beta-hydroxy butyric acid), alpha-keto acids, acetic acid and trichloroacetic acid, 1-pyrrolidone-5-carboxylic acid, capryloyl salicylic acid, alpha-hydroxy decanoic acid, alpha-hydroxy octanoic acid, gluconolactone, methoxypropyl gluconamide, oxalic acid, malic acid, tartaric acid, mandelic acid, benzylic acid, gluconic acid, benzoyl peroxide and phenol, among others, may cause the skin to become more sensitive to irritation triggered by other topically-applied chemicals such as moisturizers, sunscreens, fragrances, preservatives, surfactants (e.g. soaps, shaving cream) and other topical products. Exfoliants and other ingredients may also increase the skin's sensitivity to environmental conditions or irritants such as sunlight, wind, cold temperature and dry air, or to chemical agents such as antigens, or may exacerbate the irritation attributable to a pre-existing skin disease.

Moreover, exposure to ultraviolet (UV) radiation is known to provide a variety of undesirable effects such as photoaging and photodamage of the skin, including skin inflammation and irritation. UV radiation generally encompasses light in the wavelength range of 200-400 nm., with UVA having a wavelength of about 320-440 nm., UVB a wavelength of 290-320 nm., and UVC a wavelength of less than about 280 nm. Acute UV exposure causes solar erythema or sunburn.

Cosmetic and/or medical procedures are also known to cause an inflammatory state in the skin. Examples of such procedures are: chemical peels, microdermabrasion, laser procedures, LED light procedures, etc.

Certain skin diseases are also known to cause an inflammatory state in the skin. Examples of such skin diseases include acne, Rosacea, lupus, psoriasis, skin hypersensitivity, and/or irritant/allergic contact dermatitis.

Thus, the inflammatory skin state and/or irritation may be characterized by stinging, itching or tautness, which too may cause considerable discomfort.

WO-A-96/19184, WO-A-96/19182 and WO-A-96/19228 describe the administration of water-soluble divalent strontium, manganese, magnesium and calcium salts to reduce the irritation of compounds which have an irritant effect, in particular of exfoliants such as hydroxy acids.

U.S. Pat. No. 6,521,222 describes applying β-Diketone/hydroxylated or carbonated activated cation inorganic/organic complexes for reducing skin irritation, notably that elicited by exposure to irritant active agents/species or to the external environment.

### SUMMARY OF THE INVENTION

It is therefore an object of the present invention to provide a method for reducing an occurrence of irritation and/or inflammatory reaction in human skin.

The present invention provides a method for reducing an occurrence of irritation and/or inflammatory reaction in human skin includes applying a topical preparation to the skin. The preparation includes an amount of an agent effective to reduce an occurrence of irritation and/or inflammatory reaction in human skin. The agent includes idebenone or a derivative of idebenone.

The present invention also provides a method for reducing an occurrence of irritation and/or inflammatory reaction in human skin. The method includes:
providing a person in need of a reduction of an occurrence of irritation and/or inflammatory reaction, the occurrence of irritation and/or inflammatory reaction being responsive to an application of idebenone or a derivative of idebenone; and
applying a topical preparation to the skin, the preparation comprising an amount of an agent effective to reduce the occurrence of irritation and/or inflammatory reaction in human skin, the agent comprising idebenone or a derivative of idebenone.

Also provided by the present invention is a method for marketing a skin care product. The method includes:
providing the skin care product, the product including idebenone or a derivative of idebenone; and
providing an information indicative of a reduced potential for occurrence of irritation and/or inflammatory reaction in human skin due to a potential skin irritant through applying of the skin care product to human skin.

The present invention also provides a topical preparation for reducing an occurrence of irritation and/or inflammatory reaction in human skin due to an exfoliant applied to the skin. The topical preparation includes at least one exfoliant, and an amount of idebenone or a derivative of idebenone effective to reduce an occurrence of irritation and/or inflammatory reaction in human skin related to the exfoliant.

The inflammatory reactions and/or irritation of the skin may be due to a variety of causes, including, but not limited too, UV exposure and/or exposure to cosmetics, drugs, chemicals, cosmetic and/or medical procedures and skin disease states. For example, the inflammatory skin reaction and/or skin irritation may be due to an exposure of the skin to a potential skin irritant such as kojic acid, methyl gentisate, hydroquinone, phytic acid, retinol, retinal, retinyl palmitate, retinoic acid, tazarotene-acetylenic retinoids, and other ester derivatives of Vitamin A, Ascorbic Acid, alpha hydroxy acids and/or beta hydroxy acids. For example, the inflammatory skin reaction maybe due to a skin disease state and/or medical procedure, as discussed above.

According to an embodiment, the applying of the topical preparation may be performed after an exposure of the skin to ultraviolet radiation or other potential skin irritant. Moreover, the topical preparation may be included in a composition, such as a skin care preparation, including the potential skin irritant.

### BRIEF DESCRIPTION OF THE DRAWING

FIG. 1 shows a graph depicting percentage increase in transepidermal water loss measurements (TEWL), irritation and roughness for a retinoid alone applied to facial skin versus for idebenone plus the retinoid applied to facial skin.

### DETAILED DESCRIPTION

Idebenone (6-(10-hydroxydecyl)-2,3-dimethoxy-5-methyl-1,4-benzoquinone) is characterized by the following structural formula:

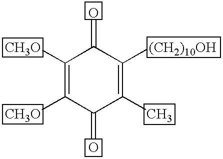

Chemical derivatives of idebenone may also be suitable for use in a method according to the present invention. Such derivatives may include, for example, esters and salts of idebenone, or protein bound forms, or other derivatives. Examples of idebenone derivatives include esters of idebenone where idebenone is esterified using glycosaminoglycans and/or their salts, for example hyaluronic acid having a molecular weight of 1 to 1,000,000 and its salts or hyaluronidase inhibitors, such as for example inter-alpha-trypsin inhibitor.

An example of a hydrophilic idebenone ester (separate synthesis) is idebenone sulphonic acid, characterized by the following structural formula:

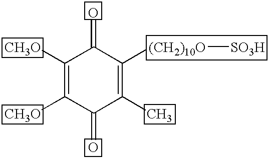

An exemplary synthesis of idebenone sulphonic acid was performed as follows: idebenone was reacted with pyridine-SO₃ and the reaction was then stopped using 1 N hydrochloric acid. After shaking out the organic phase using ethyl acetate, the organic phase was dried and concentrated under vacuum. The residue was dissolved in water and insoluble products centrifuged off. The hydrophilic idebenone ester thus recovered is suitable for application according to the invention in aqueous cosmetic and dermatological preparations.

Compositions according to the present invention may contain a concentration of idebenone or a derivative of idebenone of about 0.001-30%, 0.01-10.0%, 0.1-2.0%, or 0.5% to 1.0% by weight of the composition.

The compositions may be cosmetic, dermatologic, or pharmaceutical preparations or compositions, and may exist in a wide variety of forms, such as emulsions, suspensions, solutions and the like. In certain embodiments, the compositions are in the form of lotions, creams, gels, solutions, sprays, cleansers, powders, ointments, waxes, lipsticks, soaps, shampoos, hydroalcoholic solutions, suspensions, scrubs, saturated pads, skin or hair conditioning agents, and other types of cosmetic compositions.

For administration, the cosmetic and dermatological preparations of the invention may be applied to the skin in adequate quantity in the manner conventional for cosmetics.

Cosmetic and dermatological preparations of the invention may exist in various forms. Hence, they may be, for example a solution, an anhydrous preparation, an oil-free preparation, an emulsion or microemulsion of the type water-in-oil (W/O) or of the type oil-in-water (O/W), a multiple emulsion, for example of the type water-in-oil-in-water (W/O/W), a gel, a solid stick, an ointment or even an aerosol. It is also advantageous to administer idebenone and/or its derivatives in encapsulated form, for example in collagen matrices and other conventional encapsulation materials, for example as cellulose encapsulations, in gelatine, wax matrices or liposomally encapsulated.

It is also possible and advantageous within the scope of the present invention to add idebenone and/or its derivatives, such the sulphate of idebenone, for example, to aqueous systems or surfactant preparations for cleansing the skin.

The cosmetic and dermatological preparations of the invention may contain cosmetic auxiliaries, as are used conventionally in such preparations, for example preservatives, bactericides, perfumes, substances for preventing foaming, dyestuffs, pigments which have a coloring effect, thickening agents, surfactant substances, emulsifiers, softening, moisturizing and/or moisture-retaining substances, fats, oils, waxes or other conventional constituents of a cosmetic or dermatological formulation, such as alcohols, polyols, polymers, foam stabilizers, electrolytes, organic solvents or silicone derivatives.

In particular, idebenone and its derivatives may also be combined according to the invention with one or more traditional or other anti-oxidants and/or free-radical absorbers that are suitable or conventional for cosmetic and/or dermatological applications. Such anti-oxidants include, for example, one or more of the following: antioxidant enzymes (for example superoxide dismutase, catalase, glutathione peroxidase, glutathione S-transferase, glutathione reductase), antioxidant polyphenols such as those present in many botanical extracts (for example, green tea, white tea, black tea, licorice, grape, bilberry, mulberry, pomegranate, soy) including but not limited to flavones, isoflavones, cyanidins, anthocyanidins, catechins and related compounds (for example, chlorogenic acid, caffeic acid, ellagic acid, genistein), plant growth factors (for example N-furfuryladenine), amino acids (for example glycine, histidine, tyrosine, tryptophan) and their derivatives, imidazoles (for example urocanic acid) and their derivatives, peptides, such as D,L-camosine, D-camosine, L-camosine and their derivatives (for example anserine), carotinoids, carotenes (for example alpha-carotene, beta-carotene, lycopene) and their derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, gamma-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) and sulphoximine compounds (for example buthionine sulphoximines, homocysteine sulphoximine, buthionine sulphones, pentathionine sulphoximine, hexathionine sulphoximine, heptathionine sulphoximine) in very low, acceptable doses (for example pmole to μmoles/kg), also (metal) chelating agents (for example alpha-hydroxy fatty acids, palmitic acid, phytic acid, lactoferrin), alpha-hydroxy acids (for example citric acid, lactic acid, malic acid, mandelic acid, gluconolactone, lactobionic Acid), humic acid, colic acid, colic extracts, bilirubin, biliverdin, EDTA, EGTA and their derivatives, unsaturated fatty acids and their derivatives (for example gamma-linolenic acid, linolic acid, oleic acid), folic acid and their derivatives, ubiquinone and ubiquinol and their derivatives, vitamin C and derivatives (for example ascorbyl palmitate, Mg-ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and their derivatives, butylhydroxy toluene, butylhydroxy anisole, nordihydroguaiacic acid, nordihydroguaiaretic acid, trihydroxybutyrophenone, uric acid and its derivatives, mannose and its derivatives, sesamol, sesamolin, zinc and its derivatives (for example ZnO, ZnSO₄), selenium and its derivatives (for example selenium methionine), stilbenes and their derivatives (for example stilbene oxide, trans-stilbene oxide) and suitable derivatives (salts, esters, ethers, sugars, nucleotides, nucleosides, peptides and lipids) of these said active ingredients.

The quantity of the aforementioned anti-oxidants (one or more compounds) in the preparations may be, for example, 0.0001 wt. % to 30 wt. %, 0.05 wt. % to 20 wt. %, or 1 to 10 wt. %, based on the total weight of the preparation.

Emulsions according to the invention may contain, for example the said fats, oils, waxes and other adipoids, and water and an emulsifier, as is used conventionally for such a type of formulation.

Also any mixtures of such oil and wax components can be used advantageously within the scope of the present invention. It may also optionally be advantageous to use waxes, for example cetyl palmitate, as the single lipid component of the oil phase.

Gels according to the invention may contain alcohols of low C number, for example ethanol, isopropanol, 1,2-propane diol, glycerine and water or an above-mentioned oil in the presence of a thickening agent, which for oily-alcoholic gels is preferably silicon dioxide or an aluminium silicate, for aqueous-alcoholic or alcoholic gels is preferably a polyacrylate.

Conventional highly volatile, liquefied propellants, for example hydrocarbons (propane, butane, isobutane), which may be used alone or mixed with one another, are suitable as propellants for preparations which can be sprayed from aerosol containers according to the invention. Compressed air can also advantageously be used.

Preparations of the invention may also contain filter substances that absorb UV radiation, or sunscreens, wherein the total quantity of filter substances is, for example 0.1 wt. % to 30 wt. % or 0.5 wt. % to 10 wt. %, based on the total weight of the preparation. The preparations may also serve as sunscreen agents for the skin. Such UV filter substances include, for example, the following: avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

Preparations of the invention may also contain skin exfoliants, such as retinoids (e.g. tretinoin, retinol and retinal), carboxylic acids including alpha-hydroxy acids (e.g. lactic acid, glycolic acid), beta hydroxy acids (e.g. salicylic acid, beta-hydroxy butyric acid), alpha-keto acids, acetic acid and trichloroacetic acid, 1-pyrrolidone-5-carboxylic acid, capryloyl salicylic acid, alpha-hydroxy decanoic acid, alpha-hydroxy octanoic acid, gluconolactone, methoxypropyl gluconamide, oxalic acid, malic acid, tartaric acid, mandelic acid, benzylic acid, gluconic acid, benzoyl peroxide and phenol, among others, that may cause the skin to become more sensitive to irritation triggered by other topically-applied chemicals such as moisturizers, sunscreens, fragrances, preservatives, surfactants (e.g. soaps, shaving cream) and other topical products.

A preparation according to the invention may include, for example, by weight of the preparation:
from about 10% to about 90% of water;
from about 0% to about 20% of at least one humectant;
from about 0% to about 20% of at least one emollient;
from about 0% to about 20% of at least one ester;
from about 0% to about 10% of at least one thickener;
from about 0% to about 2% of at least one preservative;
from about 0% to about 1% of color; and
from about 0% to about 1% of fragrance.

A preparation according to the invention may include, for example, by weight of the preparation:
from about 50% to about 90% of water;
from about 1% to about 10% of glycerin;
from about 1% to about 5% of cetyl ricinoleate;
from about 1% to about 5% of isohexadecane from about 1% to about 5% of ceresin;
from about 0.5% to about 2.5% of sericin;
from about 0.1% to about 1% of glycosaminoglycans;
from about 0.1% to about 1% of dimethicone; and
from about 0.001% to about 30% of idebenone.

In the method according to the invention idebenone-containing compositions are applied to human, skin. The compositions may be applied once or more times per day depending on the activities the particular individual is engaged in. For example, an individual engaging in normal workday activities may wish to apply the compositions twice a day, once in the morning, and once in the evening, in conjunction with normal grooming. On the other hand, if the individual plans outdoor activities such as sunbathing and athletics, the compositions may be applied prior to, and during, such activities, much like a sunscreen composition is applied periodically during the day. The compositions may be used for irritation and/or inflammatory reaction on the face and neck, by applying appropriate idebenone compositions to the face and neck areas. However, the idebenone compositions may also be applied to the entire body, particularly areas which are not covered by clothing, such as the arms, neck, and lower legs.

In an embodiment of the present invention, a topical preparation directed to reducing an occurrence of irritation and/or inflammatory reaction in human skin is made available, for example for sale to wholesale or retail buyers by a wholesale or retail entity. The preparation may be made available as a product advertised or marketed to reduce irritation and/or inflammatory reaction. Also provided is information on applying the topical preparation to the skin, for example directions for use and/or indications on the product package. The topical preparation includes an amount of an agent effective to reduce an occurrence of irritation and/or inflammatory reaction in human skin. The agent includes idebenone or a derivative of idebenone.

### EXAMPLE 1

### Retinoid Irritation

In a test, idebenone was applied to the facial skin of humans. Two different test sites were setup whereby, one site was applied with idebenone twice a day for 2 weeks followed by a 2 week application period of a prescription retinoid (once a day, as prescribed). The remaining site was applied for 2 weeks just the prescription retinoid (once a day, as prescribed). Each test site was evaluated by a trained clinician for irritation and roughness using a 0 (none) to 4 (severe) scale pre and post retinoid treatment. Additionally, transepidermal water loss measurements (TEWL) were also made at the same time as visual evaluations. At the end of the treatment period, the site treated only with the prescription retinoid exhibited significant increases in TEWL, irritation and roughness compared to a moderate to minimal increase in the same parameters measured when the site was treated with Idebenone for 2 weeks prior to retinoid applications.

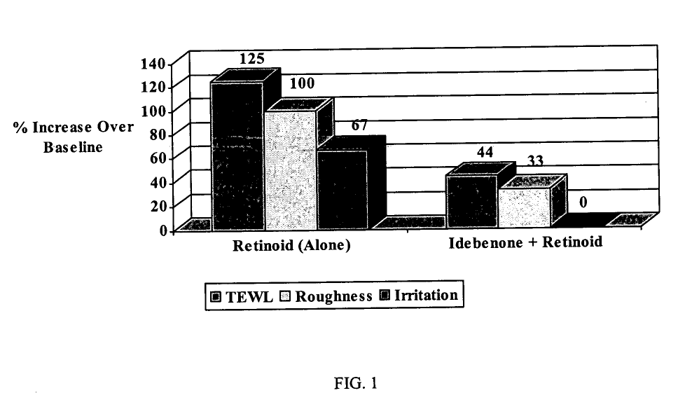

FIG. 1 graphically depicts the results of the test. The graph shows percentage increase in transepidermal water loss measurements (TEWL), irritation and roughness for a retinoid alone applied to facial skin versus for idebenone plus the retinoid applied to facial skin.

Conclusion: Idebenone significantly reduced the irritation potential produced by the prescription retinoid tested when used as a pre-treatment for 2 weeks demonstrating a method for prevention and/or reduction of inflammatory skin reactions and skin irritation in human skin.

### EXAMPLE 2

### Methyl Gentisate/Glycolic Acid Irritation

In another test, idebenone was applied to the backs of humans. Approximately 0.2 ml of a two formulas, one containing Methyl Gentisate and Glycolic Acid and the other the same combination of Methyl Gentisate and Glycolic Acid with 0.5% idebenone. Both were applied to a 2 cm×2 cm square of Webril cotton fabric (affixed to Scanpor semi-occlusive surgical tape) and placed on the back of each subject between the scapulae and waist, adjacent to the spinal mid-line. Patches were removed 24 hours after each application and the test site was allowed to rest for an additional 24 hours prior to repatching. All test sites were graded for erythema and edema prior to reapplication of the next patch. Patch testing was stopped upon the development of moderate irritation at the test sites. The combination of Methyl Gentisate and Glycolic Acid alone produced moderate irritation with edema after 1 patch in 4 out 100 subjects, whereas the combination of Methyl Gentisate and Glycolic Acid with 0.5% Idebenone allowed the completion of 9 patches to be made with only 2 subjects out of 100 demonstrating minimal irritation.

It has been found that the method of the invention provided protection against the irritation potential of the combination of Methyl Gentisate and Glycolic Acid, under exaggerated use conditions, by inhibiting irritation after patch testing.

While the invention has been described in connection with the preferred embodiment, it is not intended to limit the scope of the invention to the particular form set forth, but, on the contrary, it is intended to cover such alternatives, modifications and equivalents as may be included within the spirit and scope of the invention as defined by the appended claims.

1. A method for reducing an occurrence of irritation and/or inflammatory reaction in human skin, comprising applying a topical preparation to the skin, the preparation comprising an amount of an agent effective to reduce an occurrence of irritation and/or inflammatory reaction in human skin, the agent comprising idebenone or a derivative of idebenone.

2. The method as recited in claim 1 wherein the derivative of idebenone is selected from the group consisting of a salt of idebenone, an ester of idebenone, and a protein-bound form of idebenone.

3. The method as recited in claim 1 wherein the applying is performed after the irritation and/or inflammatory reaction has occurred.

4. The method as recited in claim 1 wherein the applying is performed prior to an exposure of the skin to a potential skin irritant.

5. The method as recited in claim 1 wherein the applying is performed concomitant with an exposure of the skin to a potential skin irritant.

6. The method as recited in claim 1 wherein the applying is performed prior to an event known or suspected to cause irritation and/or inflammatory reaction in skin.

7. The method as recited in claim 6 wherein the event is an exposure of the skin to at least one of a topically applied potential skin irritant substance, an environmental irritant, and a cosmetic or medical procedure.

8. The method as recited in claim 1 wherein the skin is at least one of facial skin, skin on the neck, skin on the arms, skin on the hands and skin on the legs.

9. The method as recited in claim 1 wherein the topical preparation further comprises an alpha-hydroxy acid or a derivative thereof.

10. The method as recited in claim 1 wherein the topical preparation further comprises a retinoid.

11. The method as recited in claim 1 wherein the topical preparation further comprises at least one of an antioxidant, an ultraviolet filter substance, an exfoliant, a surfactant, a preservative, a fragrance, a solvent and a propellant.

12. The method as recited in claim 1 wherein the applying is performed prior to an exposure of the skin to ultraviolet radiation.

13. The method as recited in claim 1 wherein the irritation and/or inflammatory reaction is due to an exposure of the skin to ultraviolet radiation.

14. The method as recited in claim 1 wherein the preparation is at least one of a cosmetic, an over-the-counter and a pharmaceutical composition.

15. The method as recited in claim 1 wherein the irritation and/or inflammatory reaction is due to an exposure of the skin to a potential skin irritant.

16. The method as recited in claim 15 wherein the potential skin irritant is selected from the group consisting of: kojic acid, methyl gentisate, hydroquinone, phytic acid, retinol, retinal, retinyl palmitate, retinoic acid, tazarotene-acetylenic retinoids, and other ester derivatives of Vitamin A, Ascorbic Acid, alpha hydroxy acids and/or beta hydroxy acids.

17. The method as recited in claim 15 wherein the potential skin irritant is included in a skin care preparation.

18. The method as recited in claim 15 wherein the topical preparation is included in a composition including the potential skin irritant.

19. The method as recited in claim 1 wherein the irritation and/or inflammatory reaction is associated with a skin condition or disease state selected from the group consisting of acne, Rosacea, lupus, psoriasis, skin hypersensitivity, irritant/allergic contact dermatitis and alike.

20. A method for reducing an occurrence of irritation and/or inflammatory reaction in human skin, comprising:
providing a person in need of a reduction of an occurrence of irritation and/or inflammatory reaction, the occurrence of irritation and/or inflammatory reaction being responsive to an application of idebenone or a derivative of idebenone; and
applying a topical preparation to the skin, the preparation comprising an amount of an agent effective to reduce the occurrence of irritation and/or inflammatory reaction in human skin, the agent comprising idebenone or a derivative of idebenone.

21. The method as recited in claim 20 wherein the derivative of idebenone is selected from the group consisting of a salt of idebenone, an ester of idebenone, and a protein-bound form of idebenone.

22. The method as recited in claim 20 wherein the applying is performed after the irritation and/or inflammatory reaction has occurred.

23. The method as recited in claim 20 wherein the topical preparation further comprises at least one potential skin irritant.

24. The method as recited in claim 20 wherein the topical preparation further comprises at least one of an antioxidant, an ultraviolet filter substance, an exfoliant, a surfactant, a preservative, a fragrance, a solvent and a propellant.

25. A method for marketing a skin care product, the method comprising:
providing the skin care product, the product including idebenone or a derivative of idebenone; and
providing an information indicative of a reduced potential for occurrence of irritation and/or inflammatory reaction in human skin due to a potential skin irritant through applying of the skin care product to human skin.

26. The method as recited in claim 25 wherein the providing an information includes providing the information in advertisements or on a packaging of the skin care product.

27. The method as recited in claim 25 wherein the applying is performed after the irritation and/or inflammatory reaction has occurred.

28. The method as recited in claim 25 wherein the derivative of idebenone is selected from the group consisting of a salt of idebenone, an ester of idebenone, and a protein-bound form of idebenone.

29. The method as recited in claim 25 wherein the potential skin irritant is a substance, and the topical preparation further includes the substance.

30. A topical preparation for reducing an occurrence of irritation and/or inflammatory reaction in human skin due to an exfoliant applied to the skin, the topical preparation comprising:
at least one exfoliant; and
an amount of idebenone or a derivative of idebenone effective to reduce an occurrence of irritation and/or inflammatory reaction in human skin related to the exfoliant.

31. The preparation as recited in claim 30 wherein the exfoliant is selected from the group consisting of a retinoid, a carboxylic acid, a beta hydroxy acid, an alpha-keto acid, acetic acid, trichloroacetic acid, 1-pyrrolidone-5-carboxylic acid, capryloyl salicylic acid, alpha-hydroxy decanoic acid, alpha-hydroxy octanoic acid, gluconolactone, methoxypropyl gluconamide, oxalic acid, malic acid, tartaric acid, mandelic acid, benzylic acid, gluconic acid, benzoyl peroxide and phenol.

32. The preparation as recited in claim 30 wherein the irritation and/or inflammatory reaction occurs upon an exposure of the skin to at least one of a topically applied potential skin irritant substance and an environmental irritant.

33. The preparation as recited in claim 30 wherein the derivative of idebenone is selected from the group consisting of a salt of idebenone, an ester of idebenone, and a protein-bound form of idebenone.
